(19) United States
(12) Patent Application Publication
(10) Pub. No.: US 2013/0030287 A1
(43) Pub. Date: Jan. 31, 2013

(54) PROXIMITY IMAGING TYPE PET APPARATUS AND SYSTEM

(71) Applicant: Taiga Yamaya, Chiba-shi (JP)

(72) Inventor: Taiga Yamaya, Chiba-shi (JP)

(73) Assignee: NATIONAL INSTITUTE OF RADIOLOGICAL SCIENCES, Chiba-shi, Chiba (JP)

(21) Appl. No.: 13/639,008

(22) PCT Filed: Apr. 8, 2010
(86) PCT No.: PCT/JP2010/056402

§ 371 Date: Oct. 2, 2012

(51) Int. Cl. A61B 6/00; G01T 1/164

(52) U.S. Cl. 600/425; 250/363.03

(57) ABSTRACT

Provided are a proximity imaging type PET apparatus and a system which include a part-specific PET scanner disposed in proximity to a specific part of a measurement target and a whole-body PET scanner which is capable of radiographing the whole body of the measurement target, the PET apparatus and system being capable of bringing PET detectors into close proximity to the specific part of the measurement target so as to ensure higher sensitivity and imaging a wide field of view.

### TECHNICAL FIELD

The present invention relates to proximity imaging type PET apparatuses and systems, and more particularly to a proximity imaging type PET apparatus and a system which are capable of bringing a PET detector into close proximity to a specific part of a measurement target so as to ensure higher sensitivity and imaging a wide field of view.

### BACKGROUND ART

The PET is a method for imaging the spatial and temporal distribution of a medicine marked by a positron emission nuclide by giving the medicine to the body, and has thus received attention as being effective for early diagnosis of whole-body cancer or Alzheimer's disease.

The PET unit is made up of radiation detectors which are disposed in an annular shape so as to surround a measurement target. The principle of the PET is as described below. Positrons emitted in the positron decay of a positron emission nuclide may disappear by annihilation in pairs with surrounding electrons and thereby a pair of annihilation radiations at 511 keV emitted substantially in diametrically opposite directions are measured with a pair of radiation detectors on the basis of the principle of coincidence. This makes it possible to identify the position of presence of the nuclide on one line segment (Line of Response: LOR) connecting between the pair of detectors.

A conventional PET unit had a degraded resolution when radiation detectors were brought into proximity to a measurement target so as to enhance the sensitivity of the scanner. Thus, the resolution has been enhanced by increasing the ring diameter of the detectors at the expense of the sensitivity. This is because of the following reason. To sufficiently detect annihilation radiations, a two-stage scheme is applicable in which the radiation is temporarily converted into visible radiation through a scintillation crystal about 3 cm in thickness and then, the resulting radiation is converted into an electrical signal by a light-receiving element such as a photomultiplier tube. However, an attempt to bring the radiation detectors into closer proximity to the body so as to enhance the sensitivity would cause deterioration, due to the thickness of the crystalline element, in the accuracy of positioning an annihilation radiation incident thereon in a diagonal direction.

To address this, a DOI (depth-of-interaction) detector has been developed which identifies the position of interaction in the depth direction within the crystal. (Patent Literatures 1-8 and Non-Patent Literatures 1-8.) Furthermore, another DOI detector has also been developed which is provided with an enhanced DOI discriminating capability using a semiconductor light-receiving element in place of a photomultiplier tube (Patent Literature 9 and Non-Patent Literature 9.) The DOI detectors can provide enhanced sensitivity and resolution at the same time because the detectors can be brought into proximity to a measurement target without deterioration in the accuracy of position detection. Note that since there is a slight shift in the angle between a pair of annihilation radiations from 180 degrees (the phenomenon called the angular deviation), it is also known that the greater the diameter of the detector ring, the greater the error in positioning the presence of the nuclide becomes. Accordingly, the radiation detector brought into close proximity to the target reduces the effects resulting from the angular deviation and contributes to further enhancement of resolution. Smaller lesion can be detected with higher resolution, while higher sensitivity can contribute to enhancing the property of equal quantity of images.

The two-layer DOI detector has been brought into practical use with a head-dedicated PET scanner “HRRT” (Non-Patent Literature 10.) Four-layer DOI detectors have also been studied and developed, for example, including the head-dedicated PET scanner “jPET-D4” developed by the inventors (Non-Patent Literature 11) or the breast cancer diagnosis dedicated PET unit (Patent Literatures 10-12 and Non-Patent Literature 12.) For the part-specific unit, since the radiation detector has a light-receiving element such as a photomultiplier tube which is not compact, the unit is big as a whole.

Furthermore, while the PET tracer is spread through the whole body, the part-specific PET unit can measure only the target part. To measure the whole body, a measurement needed to be made separately by a whole-body PET scanner before or after a measurement by the part-specific PET unit, thereby impairing temporal efficiency.

### CITATION LIST

### Patent Literature

Patent Literature 1: Japanese Patent Application Laid-Open No. Hei. 6-337289
Patent Literature 2: Japanese Patent Application Laid-Open No. Hei. 11-142523
Patent Literature 3: Japanese Patent Application Laid-Open No. 2004-132930
Patent Literature 4: Japanese Patent Application Laid-Open No. 2004-279057
Patent Literature 5: Japanese Patent Application Laid-Open No. 2007-93376
Patent Literature 6: Japanese Patent Application Laid-Open No. 2005-43062
Patent Literature 7: Japanese Patent Application Laid-Open No. Hei. 8-5746
Patent Literature 8: Japanese Patent Application Laid-Open No. Hei. 5-126957
Patent Literature 9: Japanese Patent Application Laid-Open No. 2009-121929
Patent Literature 10: Japanese Patent Application Laid-Open No. 2007-271452
Patent Literature 11: Japanese Patent Application Laid-Open No. 2007-232685
Patent Literature 12: Domestic Republication of PCT International Application No. 2007-119459
Patent Literature 13: Domestic Republication of PCT International Application No. 2009-133628

### Non-Patent Literature

Non-Patent Literature 1: J. Seidel, J. J. Vaquero, S. Siegel, W. R. Gandler, and M. V. Green, “Depth identification accuracy of a three layer phoswich PET detector module,” IEEE Trans. On Nucl. Sci., vol. 46, No. 3, pp. 485-490, June 1999
Non-Patent Literature 2: S. Yamamoto and H. Ishibashi, “ALSO depth of interaction detector for PET,” IEEE Trans. on Nucl. Sci., vol. 45, No. 3, pp. 1078-1082, June 1998
Non-Patent Literature 3: H. Liu, T. Omura, M. Watanabe, and T. Yamashita, “Development of a depth of interaction detector for γ-rays,” Nucl. Inst. Meth., A459, pp. 182-190, 2001.
Non-Patent Literature 4: N. Zhang, C. J. Thompson, D. Togane, F. Cayouette, K. Q. Nguyen, M. L. Camborde, “Anode position and last dynode timing circuits for dual-layer BGO scintillator with PS-PMT based modular PET detectors,” IEEE Trans. Nucl. Sci., Vol. 49, No. 5, pp. 2203-2207, October 2002. Non-Patent Literature 5: T. Tsuda, H. Murayama, K. Kitamura, T. Yamaya, E. Yoshida, T. Omura, H. Kawai, N. Inadama, and N. Orita, “A four layer depth of interaction detector block for small animal PET,” IEEE Trans. Nucl. Sci., vol. 51, pp. 2537-2542, October 2004.
Non-Patent Literature 6: T. Hasegawa, M. Ishikawa, K. Maruyama, N. Inadama, E. Yoshida, and H. Murayama, “Depth-of-interaction recognition using optical filters for nuclear medicine imaging,” IEEE Trans. Nucl. Sci., vol. 52, pp. 4-7, February 2005.
Non-Patent Literature 7: S. J. Hong, S. I. Kwon, M. Ito, G. S. Lee, K.-S. Sim, K. S. Park, J. T. Rhee, and J. S. Lee, “Concept verification of three-layer DOI detectors for small animal PET,” IEEE Trans. Nucl. Sci., vol. 51, pp. 912-917, June 2008.
Non-Patent Literature 8: N. Inadama, H. Murayama, M. Hamamoto, T. Tsuda, Y. Ono, T. Yamaya, E. Yoshida, K. Shibuya, and F. Nishikido, “8-layer DOI encoding of 3-dimensional crystal array,” IEEE Trans. Nucl. Sci., vol. 53, pp. 2523-2528, October 2006.
Non-Patent Literature 9: Y. Yazaki, H. Murayama, N. Inadama, A. Ohmura, H. Osada, F. Nishikido, K. Shibuya, T. Yamaya, E. Yoshida, T. Moriya, T. Yamashita, H. Kawai, “Preliminary study on a new DOI PET detector with limited number of photo-detectors,” The 5th Korea-Japan Joint Meeting on Medical Physics, Sep. 10-12, 2008, Jeju, Korea, YI-R2-3, 2008.
Non-Patent Literature 10: Wienhard K, Schmand M, Casey M E, et al: The ECATHRRT: performance and first clinical application of the new high-resolution research tomograph. IEEE Trans Nucl Sci 49: 104-110, 2002.
Non-Patent Literature 11: Yamaya T, Yoshida E, Obi T, et al: First human brain imaging by the jPET-D4prototype with a pre-computed system matrix, IEEE Trans Nucl Sci, 55: 2482-2492, 2008.
Non-Patent Literature 12: Masafumi Furuta, et al: Basic Evaluation of a C-Shaped Breast PET Scanner, 2009 IEEE Nuclear Science Symposium Conference Record, M05-1, 2009
Non-Patent Literature 13: H. Iida, et al., “A New PET Camera for noninvasive quantitation of physiological functional parametric images. HEADTOME-V-Dual.,” Quantification of brain function using PET (eds. R. Myers, V. Cunningham, D. Bailey, T. Jones) p. 57-61, Academic Press, London, 1996)

### SUMMARY OF INVENTION

### Problem to be Solved by the Invention

The present invention was developed to address the aforementioned conventional problems. It is therefore an object of the invention to provide a proximity imaging type PET apparatus and system which are capable of bringing PET detectors into close proximity to a specific part of a measurement target so as to ensure high sensitivity and imaging a wide field of view at the same time.

### Solution to Problem

The present invention was developed in accordance with the aforementioned findings and has solved the aforementioned problems by providing a proximity imaging type PET apparatus including

a part-specific PET scanner disposed in proximity to a specific part of a measurement target, and

a whole-body PET scanner capable of radiographing the whole body of the measurement target.

Here, the part-specific PET scanner can be made movable in the longitudinal direction of the measurement target relative to the whole-body PET scanner.

Furthermore, the part-specific PET scanner can be made insertable into a measurement port of the whole-body PET scanner.

Coincidence measurements can be made within the part-specific PET scanner, within the whole-body PET scanner, and by the part-specific PET scanner and the whole-body PET scanner.

Furthermore, the field of view of the part-specific PET scanner can be partially overlapped with the field of view of the whole-body PET scanner.

Furthermore, the part-specific PET scanner can be attached to a bed for the measurement target.

Furthermore, the part-specific PET scanner can be made slidable relative to the bed for the measurement target.

Furthermore, the part-specific PET scanner can be made detachable from the bed for the measurement target.

Furthermore, the part-specific PET scanner can be made attachable to the bed for the measurement target by means of a belt.

Furthermore, the part-specific PET scanner can be employed as a head PET scanner.

Furthermore, the part-specific PET scanner can be employed as a breast-dedicated PET scanner.

Furthermore, the breast-dedicated PET scanner can have cylindrically arranged detectors disposed to fit over right and left breasts.

Furthermore, the breast-dedicated PET scanner can have quadrangular-cylindrically arranged detectors disposed to fit over right and left breasts.

Furthermore, in the vicinity of a contact between the two cylindrically or quadrangular-cylindrically arranged detectors, a detector can be shared.

Furthermore, the breast-dedicated PET scanner can be employed as a single set of quadrangular-cylindrically arranged detectors so as to cover both breasts.

Furthermore, the breast-dedicated PET scanner can also be provided on the bottom thereof with a PET detector.

Furthermore, the breast-dedicated PET scanner can be configured such that a breast is sandwiched in between two planar detectors.

Furthermore, the breast-dedicated PET scanner can be configured such that right and left breasts are sandwiched in between four planar detectors, respectively.

Furthermore, with the breast-dedicated PET scanner embedded in a bed and the measurement target lying prone on the bed, the breast can be made naturally visible in the field of view of the breast PET scanner.

Furthermore, the part-specific PET scanner can be employed as a trunk-dedicated PET scanner.

Furthermore, a radiation detector which constitutes the part-specific PET scanner can be a DOI detector.

Furthermore, the light-receiving element of a radiation detector which constitutes the part-specific PET scanner and the whole-body PET scanner can be a semiconductor light-receiving element and can be used in the vicinity of an MRI apparatus or in a measurement port of the MRI apparatus.

Furthermore, the present invention provides a proximity imaging type PET apparatus system including:

a part-specific PET detector disposed in proximity to a specific part of a measurement target;

a part-specific radiation position computing unit for performing position computing based on an output from the part-specific PET detector and then outputting single event data;

a part-specific coincidence circuit for finding out two pieces of single event data which are a pair of annihilation radiations and outputting the resulting data as coincidence data;

a part-specific data collecting unit;

a part-specific image reconstruction unit for reconstructing an image based on an output from the part-specific data collecting unit;

a whole-body PET detector capable of radiographing a whole body of the measurement target;

a whole-body-specific radiation position computing unit for performing position computing based on an output from the whole-body PET detector and outputting single event data;

a whole-body-specific coincidence circuit for finding out two pieces of single event data which are a pair of annihilation radiations and outputting the resulting data as coincidence data;

a whole-body-specific data collecting unit; and

a whole-body-specific image reconstruction unit for reconstructing an image based on an output from the whole-body-specific data collecting unit, wherein

PET images from the part-specific image reconstruction unit and whole-body-specific image reconstruction unit are combined to output a composite image.

The present invention also provides a proximity imaging type PET apparatus system including:

a part-specific PET detector disposed in proximity to a specific part of a measurement target;

a part-specific radiation position computing unit for performing position computing based on an output from the part-specific PET detector and then outputting single event data;

a part-specific coincidence circuit for finding out two pieces of single event data which are a pair of annihilation radiations and outputting the resulting data as coincidence data;

a part-specific data collecting unit;

a whole-body PET detector capable of radiographing a whole body of the measurement target;

a whole-body-specific radiation position computing unit for performing position computing based on an output from the whole-body PET detector and outputting single event data;

a whole-body-specific coincidence circuit for finding out two pieces of single event data which are a pair of annihilation radiations and outputting the resulting data as coincidence data;

a whole-body-specific data collecting unit; and

an image reconstruction unit for reconstructing an image based on outputs from the part-specific data collecting unit and the whole-body-specific data collecting unit.

The present invention also provides a proximity imaging type PET apparatus system including:

a part-specific PET detector disposed in proximity to a specific part of a measurement target;

a part-specific radiation position computing unit for performing position computing based on an output from the part-specific PET detector and then outputting single event data;

a whole-body PET detector capable of radiographing a whole body of the measurement target;

a whole-body-specific radiation position computing unit for performing position computing based on an output from the whole-body PET detector and outputting single event data;

a coincidence unit for finding out two pieces of single event data, which are a pair of annihilation radiations, from data into which pieces of single event data provided by the part-specific radiation position computing unit and the whole-body-specific radiation position computing unit are combined, and outputting the resulting data as coincidence data;

a data collecting unit; and

an image reconstruction unit for reconstructing an image based on an output from the data collecting unit.

The present invention also provides a proximity imaging type PET apparatus system including:

a part-specific PET detector disposed in proximity to a specific part of a measurement target;

a part-specific radiation position computing unit for performing position computing based on an output from the part-specific PET detector and then outputting single event data;

a part-specific data collecting unit for saving the single event data;

a whole-body PET detector capable of radiographing a whole body of the measurement target;

a whole-body-specific radiation position computing unit for performing position computing based on an output from the whole-body PET detector and outputting single event data;

a whole-body-specific data collecting unit for saving the single event data;

a coincidence unit for finding out two pieces of single event data, which are a pair of annihilation radiations, from data into which pieces of single event data provided by the part-specific data collecting unit and the whole-body-specific data collecting unit are combined, and outputting the resulting data as coincidence data; and

an image reconstruction unit for reconstructing an image based on an output from the coincidence unit.

The present invention also provides a proximity imaging type PET apparatus system including:

a part-specific PET detector disposed in proximity to a specific part of a measurement target;

a part-specific radiation position computing unit for performing position computing based on an output from the part-specific PET detector and then outputting single event data;

a whole-body PET detector capable of radiographing a whole body of the measurement target;

a whole-body-specific radiation position computing unit for performing position computing based on an output from the whole-body PET detector and outputting single event data;

a data collecting unit for combining and saving the two types of single event data;

a coincidence unit for finding out, from combined data, two pieces of single event data which are a pair of annihilation radiations and outputting the resulting data as coincidence data; and

an image reconstruction unit for reconstructing an image based on an output from the coincidence unit.

### Advantageous Effects of Invention

Taking a cancer diagnosis as an example, while a specific part is being examined with high accuracy, it can be checked at once whether the cancer has been metastasized to the whole body.

### BRIEF DESCRIPTION OF DRAWINGS

FIG. 1 shows (a) a front view and (b) a side view, each illustrating a first embodiment of the present invention.

FIG. 2 shows (a) a front view and (b) a plan view, each illustrating another form of guide rails and a sliding mechanism of a head-dedicated PET scanner.

FIG. 3 shows a typical operative aspect of a bed.

FIG. 4 is a block diagram illustrating various structural aspects of a system.

FIG. 5 is a view illustrating the sensitivity profile of PET images along the longitudinal axis of a measurement target.

FIG. 6 is a cross-sectional view illustrating the positional relationship between a head-dedicated PET detector and a whole-body PET detector at the start of a measurement.

FIG. 7 is a perspective view illustrating another embodiment which enables a head-dedicated PET scanner to be removed.

FIG. 8 is an exploded perspective view illustrating a part-specific (organ dedicated) PET scanner which can be used for other than the head.

FIG. 9 shows (a) a front view and (b) a sectional side view, each illustrating a second embodiment of the present invention.

FIG. 10 shows (a) a front view and (b) a sectional side view, each illustrating a third embodiment of the present invention.

FIG. 11 shows plan views illustrating various examples of the part-specific PET detectors according to the second and third embodiments.

FIG. 12 shows (a) a front view and (b) a sectional side view, each illustrating an example of a PET/MRI scanner to which the present invention is applied.

FIG. 13 is a side view illustrating the travelling states from the start to the end of an examination in the aforementioned example.

### DESCRIPTION OF EMBODIMENTS

Now, the present invention will be described in more detail below with reference to the drawings in accordance with the embodiments.

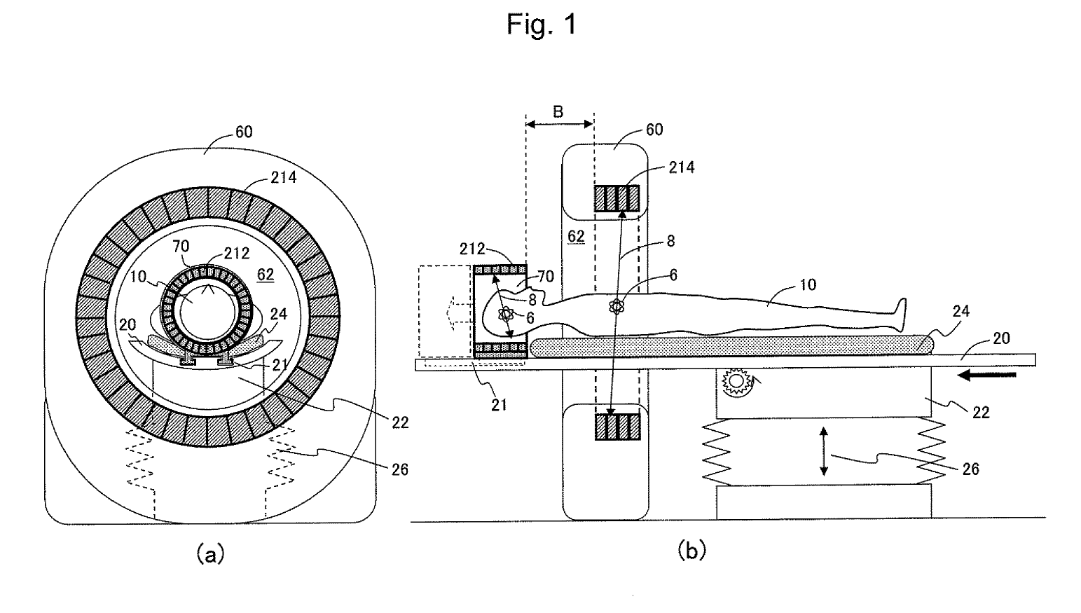

FIG. 1 shows an embodiment of the present invention. Shown in the figure is a scannerwhole-body PET scanner 60 which is of a conventional type or one that is similar in structure thereto and in which a bed moving scanner 22 slides and inserts a measurement target 10 (for example, patient) on a bed 20 together into a patient port 62 of the scannerwhole-body PET scanner 60, whereby measurements in a wider range than the width of the field of view of an embedded PET detector 214 can be achieved. Shown here are an example of a positron emission nuclide 6, an example of annihilation radiation 8, a cushion 24 for protecting the patient 10, and a bed up-and-down mechanism 26.

FIG. 1 shows an example of a head-dedicated PET scanner 70 integrated with the bed 20. The head-dedicated PET scanner 70 includes a PET detector 212, which is preferably a DOT detector in order to be brought into close proximity to the measurement target. Furthermore, the outer diameter of the head-dedicated PET scanner 70 has to be made less than the inner diameter of the patient port 62 so that the PET scanner 70 can be inserted into the patient port 62. Candidates for compact DOI detectors may include a DOI detector which is being developed by the inventors as disclosed in Patent Literature 9 and Non-Patent Literature 9 (hereinafter referred to as the crystal cube detector.)

The head-dedicated PET scanner 70 may be secured to the bed 20, but in FIG. 1, the head-dedicated PET scanner 70 is made slidable relative to the bed 20 with guide rails 21 provided on the bed 20. When the measurement target 10 is laid on the bed 20, this structure can facilitate a set-up of the measurement target 10 by removing the head-dedicated PET scanner 70 to the left in the figure (in the direction shown by a dotted line arrow in the figure.)

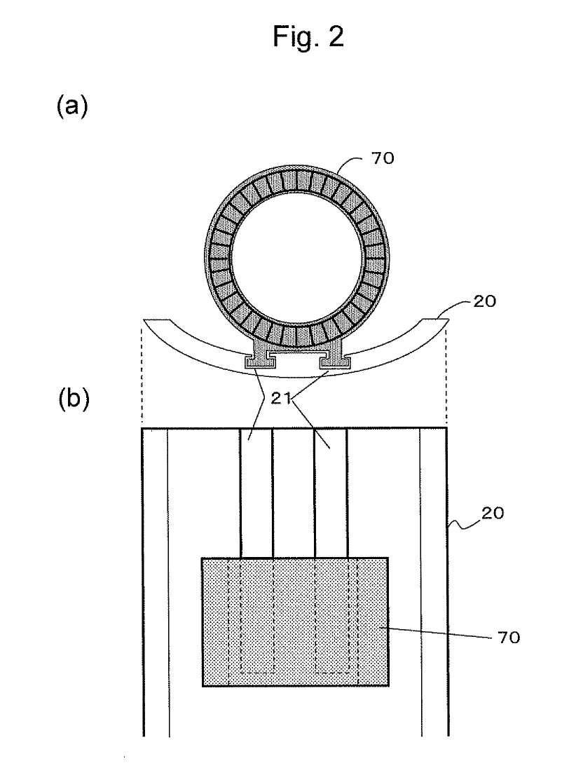

FIG. 2 shows another form of the guide rails 21 and the sliding mechanism of the head-dedicated PET scanner 70, in which one end of the guide rails 21 is extended to the end of the bed 20, thereby making the head-dedicated PET scanner 70 removable.

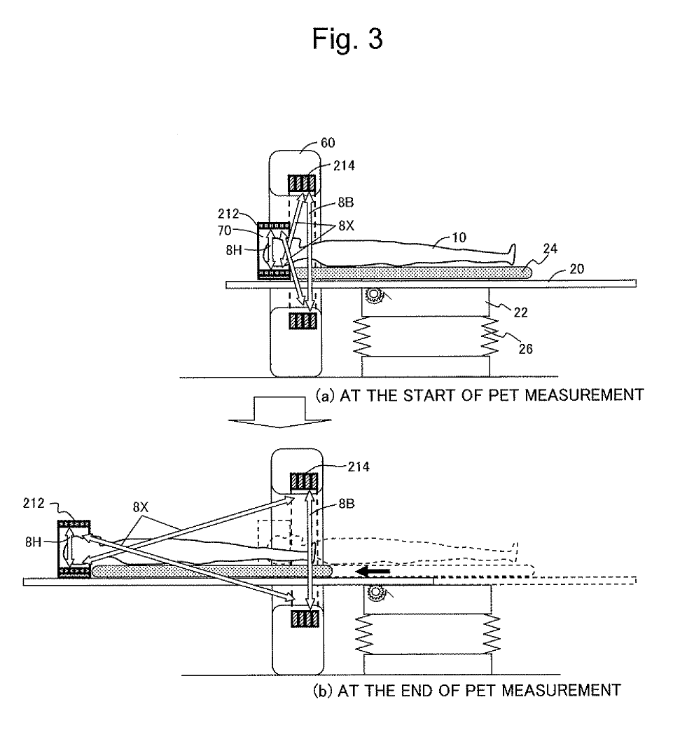

FIG. 3 shows a typical operational example of the bed 20 of FIG. 1. In the figure, the position at which the field of view of a head-dedicated PET detector 212 and the field of view of a whole-body PET detector 214 are in contact with each other is defined as (a) the start of a PET measurement, whereas the position at which the distal end of a measurement range (in the figure, the toe of the measurement target 10) has come into the field of view of the whole-body PET detector 214 is defined as (b) the end of a PET measurement.

Note that the start position and the end position may be exchangeable, or alternatively a reciprocating motion can also be employed. The start position and the end position do not have to be defined in a strict sense.

Furthermore, the bed 20 may be moved continuously or in a step and shoot manner.

The movement of the bed 20 can be stopped when a specific part has come into the field of view of the whole-body PET detector 214. In this case, not a local part (the head in the figure) and the whole body, but two parts, i.e., the head and another local part, can be examined with high accuracy. This is shown in a previous example. At the Research Institute for Brain and Blood Vessels—Akita, studies were conducted by arranging two commercially available PET scanners side by side in order to PET radiograph brain and heart regions at the same time independently of each other (Non-Patent Literature 13).

If another local part is wider than the width of the field of view of the whole-body PET detector 214, then the bed 20 may be moved by the amount that allows for covering the another local part.

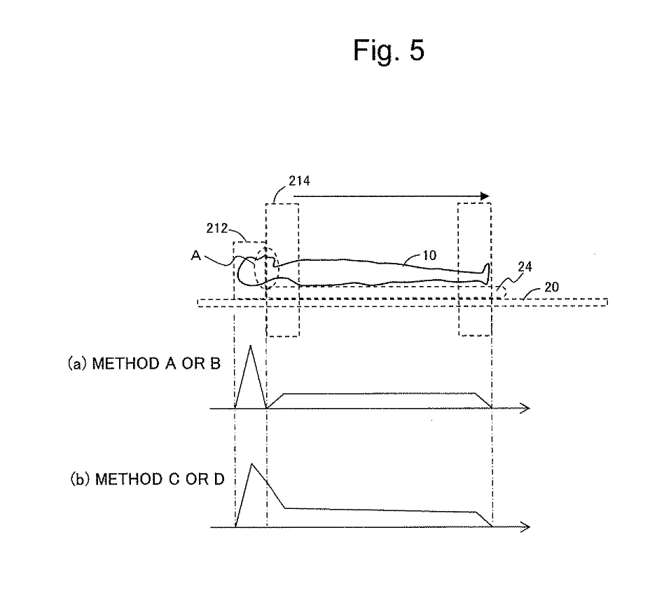

Now, coincidence measurements will be described. As shown in FIG. 3, it is necessary to make at least a coincidence measurement 8H with the head-dedicated PET detectors 212 and a coincidence measurement 8B with the whole-body PET detectors 214. Furthermore, the method with an open. PET scanner disclosed in Patent Literature 13 can also be applied to allow both the detectors to make a coincidence measurement 8X. This can ensure that annihilation radiations occurring in the vicinity A of the boundary (see FIG. 5 to be referred to later) between the head-dedicated detector 212 and the whole-body PET detector 214 and in the gap B (FIG. 1(b)) therebetween will be detected without fail.

In practice, as shown in FIG. 3(b), with the head-dedicated PET detector 212 and the whole-body PET detector 214 sufficiently separated from each other, a diagonal line of response with an increased length crossing the measurement target causes the measurement target to have the effects of absorption and scattering. Therefore, lines of response inclined to some extent may include many noise components, and can thus also be left unmeasured or unused in the image reconstruction computing.

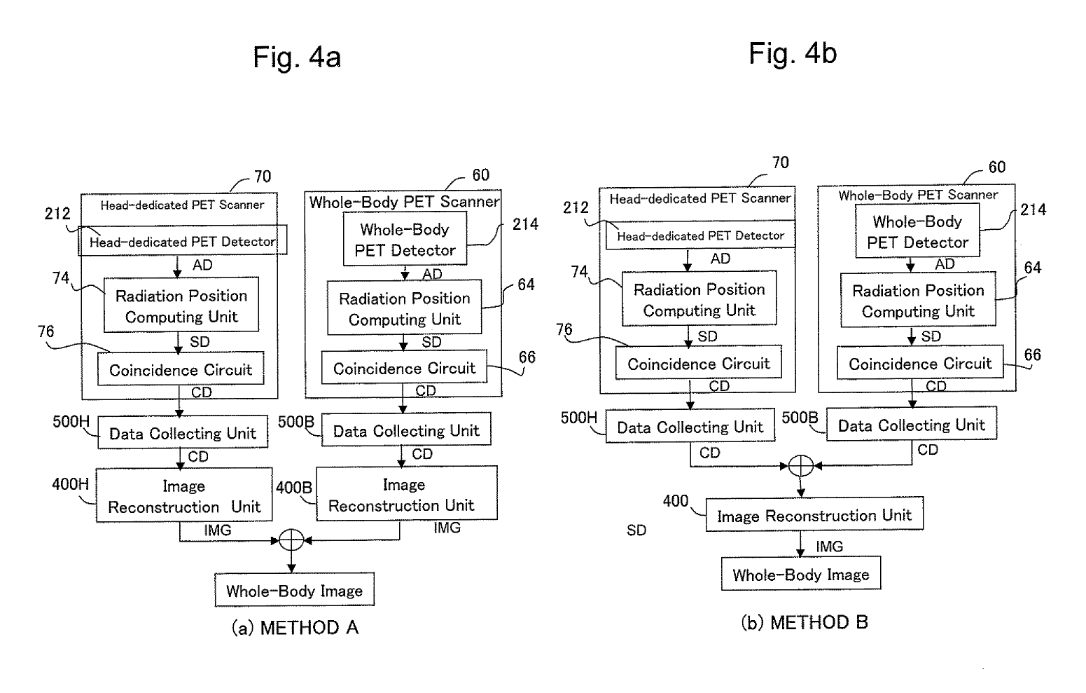

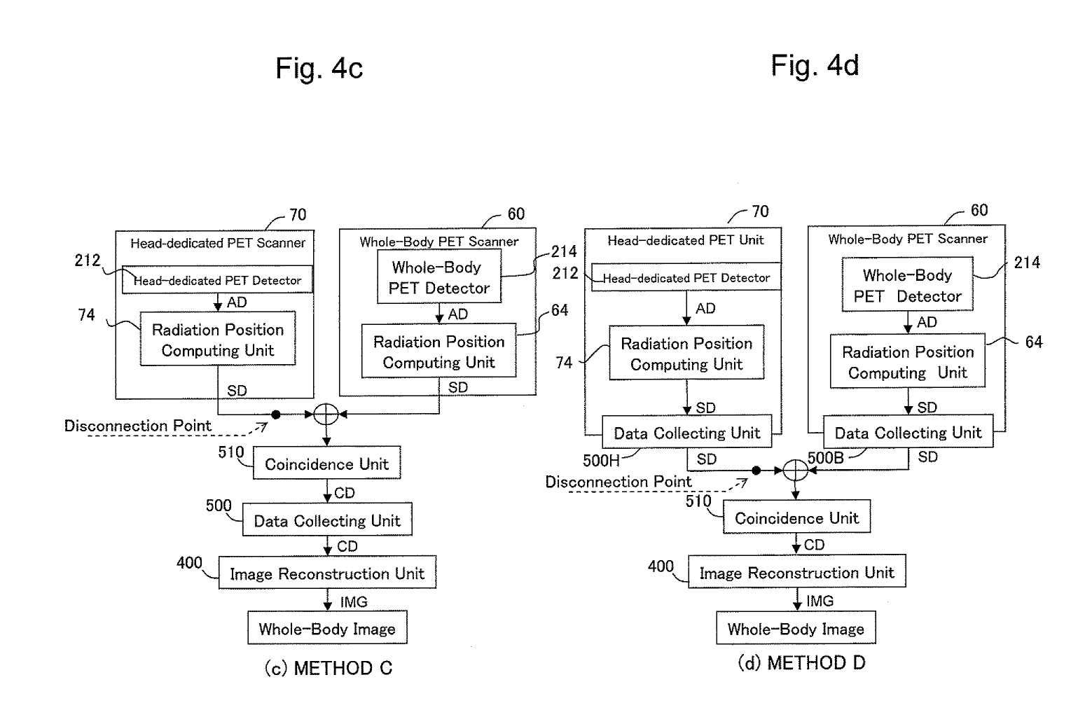

FIG. 4 shows the configuration of the system, and FIG. 5 shows the sensitivity profile of PET images along the longitudinal axis of the measurement target. Here, the whole-body PET detector 214 is to cover the entire field of view except for the field of view that is covered by the head-dedicated PET detector 212.

First, referring to FIG. 4, a description will be made to the basic configuration of the system. In the head-dedicated PET scanner 70, one of annihilation radiations having been detected by the head-dedicated PET detector 212 is sent to a head-specific radiation position computing scanner 74 as analog data AD, which is then subjected to position computing and digital processing and sent to a head-specific coincidence circuit 76 as single event data SD. The head-specific coincidence circuit 76 finds out two pieces of the single event data SD, which are a pair of annihilation radiations and then sent as coincidence data CD to a head-specific data collecting scanner 500H. Then, a head-specific image reconstruction scanner 400H performs image reconstruction computing to output a PET image IMG. The basic configuration of the whole-body PET scanner 60 is the same as that of the head-dedicated PET scanner 70. However, since a wider range of the measurement target is measured while the bed 20 is being moved, the information on the relative position between the measurement target and the whole-body PET detector 214 has to be associated with the coincidence data CD.

The method A shown in FIG. 4(a) is an example in which the head-dedicated PET scanner 70 and the whole-body PET scanner 60 each form an independent system, and the final respective PET images are combined into a composite image (a whole-body image when the whole body except the head is measured by the whole-body PET scanner).

However, the coincidence measurement between the whole-body PET detector 214 and the head-dedicated PET detector 212 as shown as 8X in relation to FIG. 3 cannot be made with this configuration, causing an extreme degradation in sensitivity at the interface between the head and the torso as shown in FIG. 5(a).

In the method B shown in FIG. 4(b), an image reconstruction unit 400 can be shared, and the coincidence data CD from the head-specific data collecting unit 500H and a whole-body-specific data collecting unit 500B can be combined. However, it is not possible to make coincidence measurements between the whole-body PET-detector 214 and the head-dedicated PET detector 212.

In the method C of FIG. 4(c) and the method D of FIG. 4(d), the systems are configured to enable coincidence measurements between the whole-body PET detector 214 and the head-dedicated PET detector 212. In the method C of FIG. 4(c), the radiation position computing unit 74 and a radiation position computing unit 64 each deliver the single event data SD, which are combined and then sent to a shared coincidence unit 510. In the method D of FIG. 4(d), in the respective units, the single event data SD is saved temporarily in the head-specific data collecting unit 500H or the whole-body-specific data collecting unit 500B, and then the shared coincidence unit 510 searches for coincidence pairs. In the method C of FIG. 4(c), the processing up to that conducted by the coincidence unit 510 can be accomplished online at high speeds. On the other hand, since the wiring, illustrated as the disconnection point in the figure, between the head-specific radiation position computing unit 74 and the coincidence unit 510 is complicated, the head-dedicated PET scanner 70 cannot be removed with ease. Therefore, the method C can be said to be suitable for a system with a part-specific PET scanner (here, the head-dedicated PET scanner 70) and the whole-body PET scanner 60 being integrated with each other from the beginning. On the other hand, the method D of FIG. 4(d) can ensure only a lower online capability than the method C does because the former method allows the data collecting units 500H and 500B to temporarily save the single event data SD. However, since the wiring, illustrated as the disconnection point in the figure, between the head-specific data collecting unit 500H and the coincidence unit 510 can be formed in a simple structure, for example, of LAN cables, a part-specific PET scanner (here, the head-dedicated PET scanner 70) and the whole-body PET scanner 60 can be separated from each other with ease.

In the method C of FIG. 4(c) and the method D of FIG. 4(d), coincidence measurements can be made between the whole-body PET detector 214 and the head-dedicated PET detector 212. It is thus possible to prevent degradation in sensitivity in the vicinity of the boundary between the head and the torso as shown in FIG. 5(b).

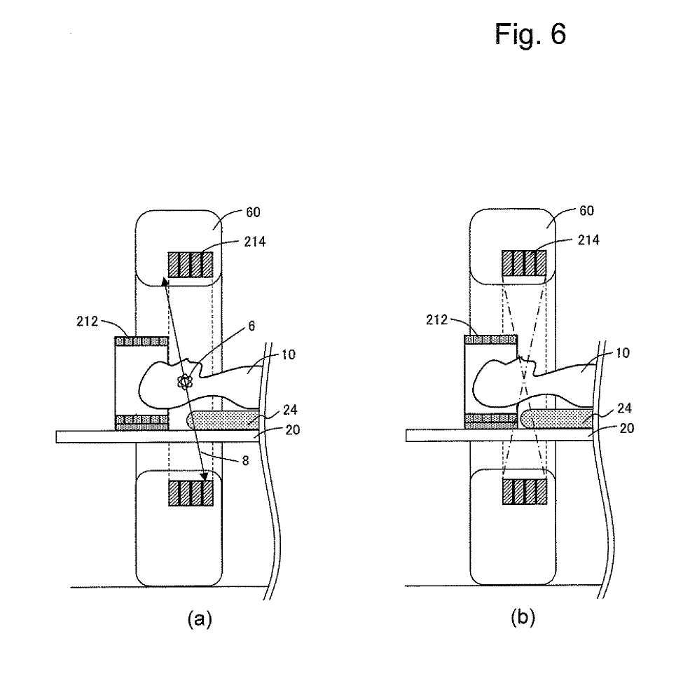

FIG. 6 shows the positional relationship between the head-dedicated PET detector 212 and the whole-body PET detector 214 at the start of a measurement. In FIG. 6(a), the position at which the field of view of the head-dedicated PET detector 212 and the field of view of the whole-body PET detector 214 are in contact with each other is defined as the start of a PET measurement. However, like an annihilation radiation 8 illustrated, the radiation is missed in the gap between the head-dedicated PET detector 212 and the whole-body PET detector 214.

In this context, as illustrated in FIG. 6(b), the fields of view of the head PET detector 212 and the whole-body PET detector 214 may be slightly overlapped with each other. In FIG. 6(b), the position at which a line segment (denoted by a broken line in the figure) connecting between the farthest detectors on the ring ends of the whole-body PET detectors 214 is in contact with the head PET detector 212 is defined as the start of a PET measurement.

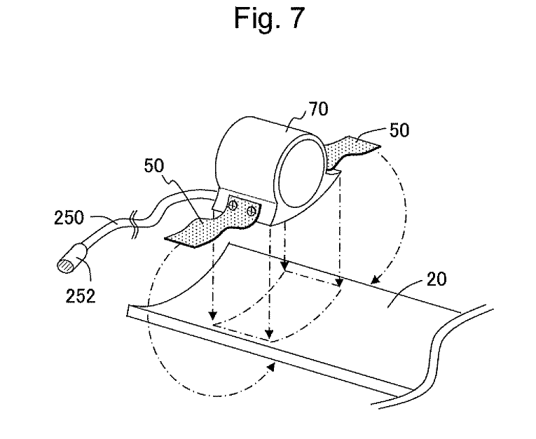

FIG. 7 shows another form which enables the head PET scanner 70 to be removed. Reference numeral 50 denotes a belt with which the head PET scanner 70 is secured to the bed 20. For example, Magic Tape (trade mark) can facilitate fixation and release with ease. Also shown are a signal and power supply cable 250 and a terminal 252. This embodiment requires no special mechanism for the bed 20, and advantageously allows the head PET scanner to be attached with ease to any existing bed.

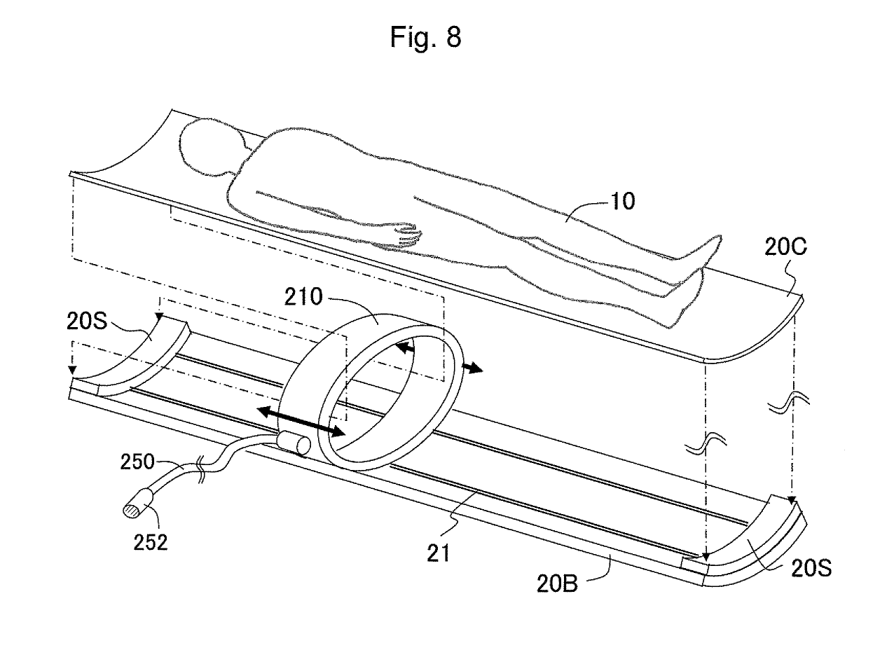

Note that the scannerpart-specific (organ dedicated) scanners are not limited to the head-specific scanner. FIG. 8 shows a scannerpart-specific (organ dedicated) scanner that can be used for other than the head. A detector ring 210 can be as big as the whole body can pass therethrough and is not necessarily circular. The figure shows an elliptical one. The bed 20 is formed of a base 20B including the guide rails 21, a support 20S, and a cover 20C, while a detector ring 210 is disposed allowing part of the ring to be sandwiched between the base 20B and the cover 20C. This advantageously allows the detector ring 210 to slidably move to an appropriate position, at which a measurement point is covered, while the measurement target 10 is kept lying on the bed 20.

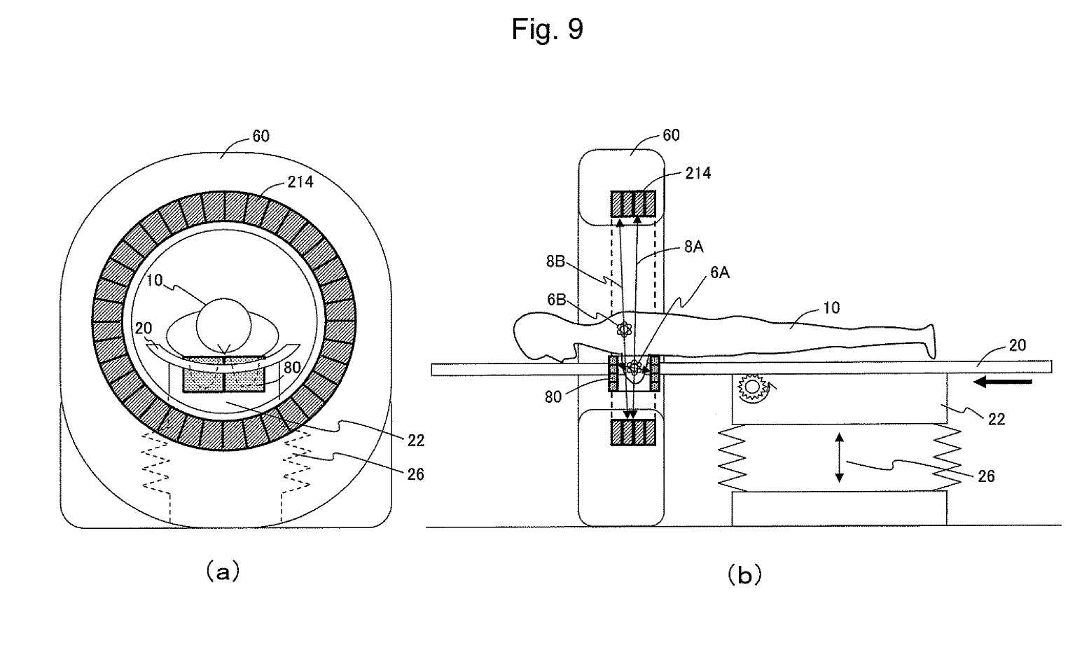

FIG. 9 shows a second embodiment in which a breast-dedicated part-specific PET detector 80 is integrated with the bed 20 and combined with the whole-body PET scanner 60, whereby the whole body is examined at the same time while the breast part is being thoroughly examined. The breast-dedicated part-specific PET detector 80 is configured to fit over the breast with the measurement target lying prone on the bed. The bottom of the breast-dedicated part-specific PET detector 80 may be covered with a detector, but illustrated to be opened. FIG. 9 shows the part-specific PET detector 80 which has just come into the field of view of the whole-body PET detector 214. With the bottom of the breast-dedicated part-specific PET detector 80 opened, an annihilation radiation 8B originated from a positron emission nuclide other than the breast, e.g., shown at 6B can be measured by the whole-body PET detector 214 without being blocked by the part-specific PET detector 80. Furthermore, for example, of annihilation radiations originated from a positron emission nuclide shown at 6A within the breast, the annihilation radiation, which is shown at 8A and has occurred in the direction in which the part-specific PET detector 80 cannot detect the event, can be measured by the whole-body PET detector 214. In this case, since a coincidence measurement does not necessarily need to be made between the part-specific PET detector 80 and the whole-body PET detector 214, the system can be configured to employ any one of the methods A to D shown in FIG. 4.

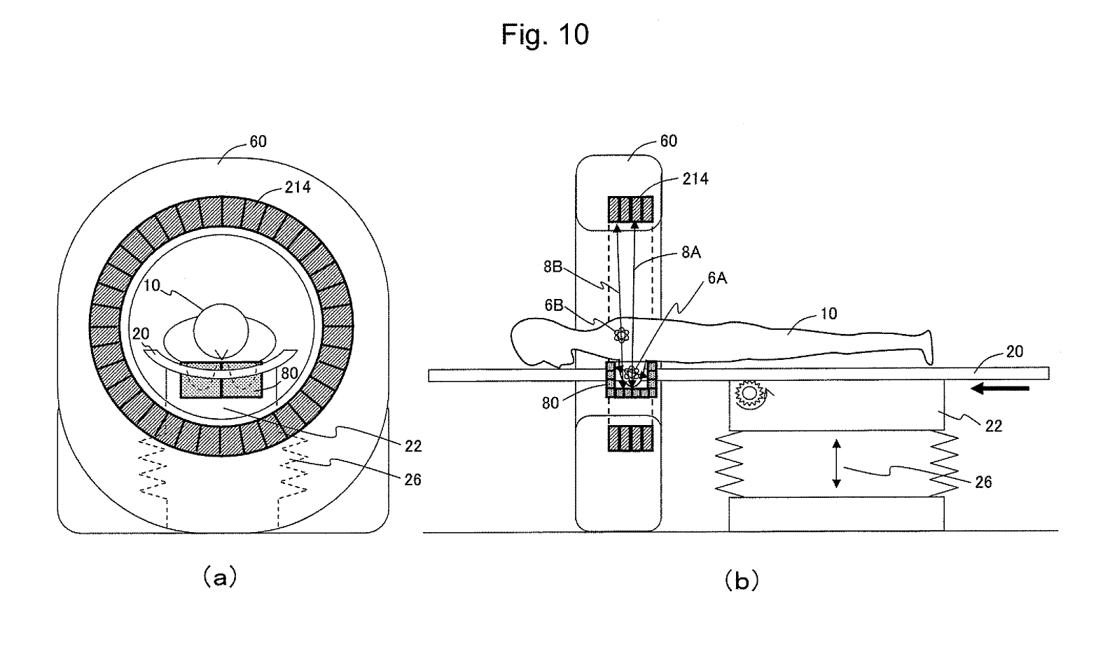

On the other hand, FIG. 10 shows a third embodiment in which the breast-dedicated part-specific PET detector 80 is provided on the bottom thereof with a detector. For example, the annihilation radiation 8B originated from a positron emission nuclide as shown at 6B located at other than the breast can be coincidence measured by the part-specific PET detector 80 and the whole-body PET detector 214. Furthermore, for example, of annihilation radiations originated from a positron emission nuclide shown at 6A within the breast, the annihilation radiation shown at 8A can also be coincidence measured by the part-specific PET detector 80 and the whole-body PET detector 214. In this case, since a coincidence measurement has to be made between the part-specific PET detector 80 and the whole-body PET detector 214, the system has to be configured to employ the method C or D shown in FIG. 4.

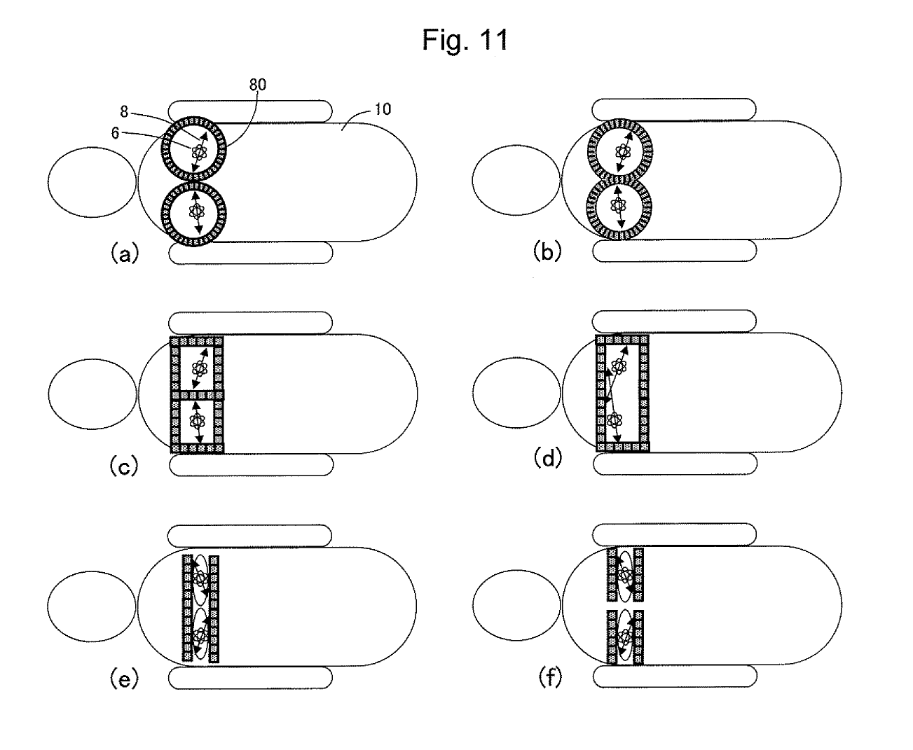

FIG. 11 shows the breast-dedicated part-specific PET detector 80, which has been depicted in FIG. 9 or FIG. 10, with detector arrangements illustrated as viewed from another angle. FIG. 11(a) shows detectors disposed cylindrically to fit over the right and left breasts, and FIG. 11(b) shows a modified example of FIG. 11(a), with a detector being shared in the vicinity of the contact between the two cylinders. FIG. 11(c) shows detectors arranged in a quadrangular cylindrical shape and disposed to fit over the right and left breasts, and FIG. 11(d) shows a similar quadrangular cylindrical shape but disposed to fit over the right and left breasts altogether. FIG. 11(e) shows a mode with two planar PET detectors sandwiching the breasts, and FIG. 11(f) shows a modified mode of FIG. 11(e), with the detector separated into two each to fit over each of the right and left breasts.

Note that the bed 20 may be configured not to slide, but the bed 20 may be fixed with the whole-body PET scanner 60 allowed to slide.

### Example

The present invention was applied to a PET/MRI apparatus as shown in an example below.

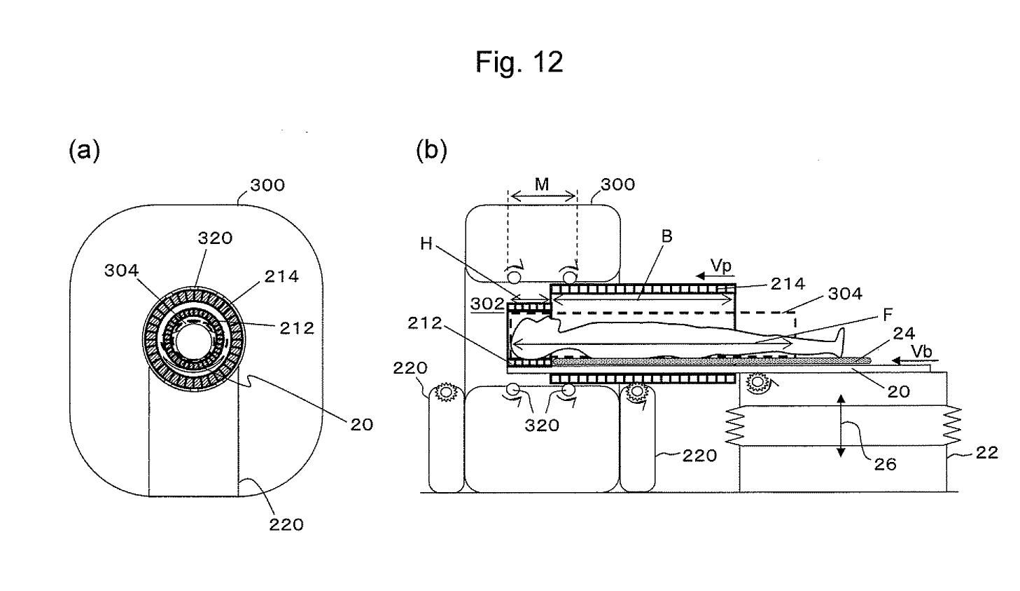

As shown in FIG. 12(a) (front view) and (b) (side cross-sectional view), the example includes an MRI apparatus 300 having a measurement port (here, a patient port) 302, the whole-body PET detector 214 having an outer diameter less than the inner diameter of the patient port 302, and the head PET detector 212 having an outer diameter less than the inner diameter of the whole-body PET detector 214. The head PET detector 212 is secured to the bed 20, while the whole-body PET detector 214 is made movable by a PET detector moving unit 220 independently of the bed 20 in the horizontal direction. The figure shows rollers 320 for supporting the PET detector 214 within the patient port 302, and the whole-body PET detector moving unit 220.

The PET field of view expressed by the head field of view H+the torso field of view B is wider than the effective measurement field of view M of the MRI apparatus 300 (referred to as the MRI field of view), and the head-dedicated PET detector 212 and the whole-body PET detector 214 are slid at different speeds, whereby a field of view F much wider than the PET field of view can be captured substantially at the same time by the PET and MRI. Here, it is assumed that the head PET detector 212 and the bed 20 are integrated to slide at speed Vb, while the torso PET detector 214 slides at speed Vp.

The figure shows an RF coil 304 for the MRI apparatus 300. The portion of the RF coil 304 on the back of a patient may be integrated with a cushion 24.

As the PET detectors 212 and 214, it is possible to employ those that operate with stability under the MRI magnetic field environment, for example, semiconductor light-receiving elements such as APDs in place of the photomultiplier tube or the aforementioned crystal cube detector.

The RF coil 304 is provided so as to cover substantially the entire field of view of the body axis in the same manner as the PET field of view P. The RF coil 304 is installed inwardly (inside the inner diameter) of the PET detectors 212 and 214 because a higher signal S/N ratio is available when installed in closer proximity to the patient 10 as well as in order to avoid electrical noise from the PET detectors 212 and 214. Note that since the annihilation radiation tends to easily pass through the RF coil, the presence of the RF coil 304 has limited effects on PET measurements.

Note that the bed 20 can be moved by the bed moving unit 22 at a constant speed or in a step and shoot manner.

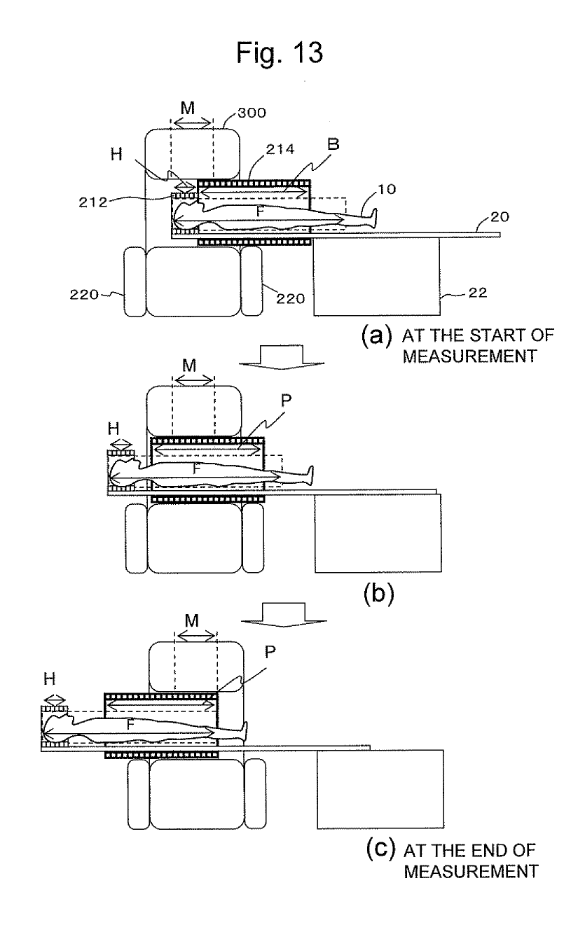

The travelling state from the start to the end of an examination is shown in FIG. 13. Here, assuming that both the bed travel speed Vb and the PET detector travel speed Vp are constant, and the MRI measurement time=the PET measurement time=T, then Vp and Vb are expressed by the equations below.

Vp=(B+H−M)/T (1)

Vb=(F−M)/T (2)

### INDUSTRIAL APPLICABILITY

The present invention is useful as a proximity imaging type PET apparatus and system which are capable of bringing PET detectors into close proximity to a specific part of a measurement target so as to ensure high sensitivity and imaging a wide field of view.

### REFERENCE SIGNS LIST

6 . . . positron emission nuclide
8 . . . annihilation radiation
10 . . . patient (measurement target)
20 . . . bed
21 . . . guide rail
22 . . . bed moving unit
26 . . . bed up-and-down mechanism
50 . . . securing belt
60 . . . whole-body PET scanner
62 . . . patient port of the whole-body PET scanner (measurement port)
64, 74 . . . radiation position computing unit
66, 76 . . . coincidence circuit
70 . . . head-dedicated PET scanner
80 . . . breast-dedicated PET detector
212 . . . head-dedicated PET detector
214 . . . whole-body PET detector
220 . . . whole-body PET detector moving unit
400 . . . image reconstruction unit
500 . . . data collecting unit
510 . . . coincidence unit
B . . . torso PET field of view
H . . . head-dedicated PET field of view

1. A proximity imaging type PET apparatus, comprising:
a part-specific PET scanner disposed in proximity to a specific part of a measurement target; and
a whole-body PET scanner capable of radiographing a whole body of the measurement target.

2. The proximity imaging type PET apparatus according to claim 1, wherein the part-specific PET scanner is made movable in a longitudinal direction of the measurement target relative to the whole-body PET scanner.

3. The proximity imaging type PET apparatus according to claim 2, wherein the part-specific PET scanner is made insertable into a measurement port of the whole-body PET scanner.

4. The proximity imaging type PET apparatus according to claim 1, wherein coincidence measurements are made within the part-specific PET scanner, within the whole-body PET scanner, and by the part-specific PET scanner and the whole-body PET scanner.

5. The proximity imaging type PET apparatus according to claim 1, wherein a field of view of the part-specific PET scanner is partially overlapped with a field of view of the whole-body PET scanner.

6. The proximity imaging type PET apparatus according to claim 1, wherein the part-specific PET scanner is attached to a bed for the measurement target.

7. The proximity imaging type PET apparatus according to claim 6, wherein the part-specific PET scanner is made slidable relative to the bed for the measurement target.

8. The proximity imaging type PET apparatus according to claim 6, wherein the part-specific PET scanner is made detachable from the bed for the measurement target.

9. The proximity imaging type PET apparatus according to claim 8, wherein the part-specific PET scanner is made attachable to the bed for the measurement target by means of a belt.

10. The proximity imaging type PET apparatus according to claim 1, wherein the part-specific PET scanner is a head-dedicated PET scanner.

11. The proximity imaging type PET apparatus according to claim 1, wherein the part-specific PET scanner is a breast-dedicated PET scanner.

12. The proximity imaging type PET apparatus according to claim 11, wherein the breast-dedicated PET scanner has cylindrically arranged detectors disposed to fit over right and left breasts.

13. The proximity imaging type PET apparatus according to claim 11, wherein the breast-dedicated PET scanner has quadrangular-cylindrically arranged detectors disposed to fit over right and left breasts.

14. The proximity imaging type PET apparatus according to claim 12, wherein in the vicinity of a contact between the two cylindrically or quadrangular-cylindrically arranged detectors, a detector is shared.

15. The proximity imaging type PET apparatus according to claim 11, wherein the breast-dedicated PET scanner is a single set of quadrangular-cylindrically arranged detectors so as to cover both breasts.

16. The proximity imaging type PET apparatus according to claim 11, wherein the breast-dedicated PET scanner is also provided on the bottom thereof with a PET detector.

17. The proximity imaging type PET apparatus according to claim 11, wherein the breast-dedicated PET scanner is configured such that a breast is sandwiched in between two planar detectors.

18. The proximity imaging type PET apparatus according to claim 11, wherein the breast-dedicated PET scanner is configured such that right and left breasts are each sandwiched in between four planar detectors, respectively.

19. The proximity imaging type PET apparatus according to claim 11, wherein the breast-dedicated PET scanner is embedded in a bed, and when the measurement target lies prone on the bed, a breast is made naturally visible in the field of view of the breast-dedicated PET scanner.

20. The proximity imaging type PET apparatus according to claim 1, wherein the part-specific PET scanner is a trunk-dedicated PET scanner.

21. The proximity imaging type PET apparatus according to claim 1, wherein a radiation detector which constitutes the part-specific PET scanner is a DOI detector.

22. The proximity imaging type PET apparatus according to claim 1, wherein a light-receiving element of a radiation detector which constitutes the part-specific PET scanner and the whole-body PET scanner is a semiconductor light-receiving element and is used in the vicinity of an MRI apparatus or in a measurement port of the MRI apparatus.

23. A proximity imaging type PET apparatus system, comprising:
a part-specific PET detector disposed in proximity to a specific part of a measurement target;
a part-specific radiation position computing unit for performing position computing based on an output from the part-specific PET detector and then outputting single event data;
a part-specific coincidence circuit for finding out two pieces of single event data which are a pair of annihilation radiations and outputting the resulting data as coincidence data;
a part-specific data collecting unit;
a part-specific image reconstruction unit for reconstructing an image based on an output from the part-specific data collecting unit;
a whole-body PET detector capable of radiographing a whole body of the measurement target;
a whole-body-specific radiation position computing unit for performing position computing based on an output from the whole-body PET detector and outputting single event data;
a whole-body-specific coincidence circuit for finding out two pieces of single event data which are a pair of annihilation radiations and outputting the resulting data as coincidence data;
a whole-body-specific data collecting unit; and
a whole-body-specific image reconstruction unit for reconstructing an image based on an output from the whole-body-specific data collecting unit, wherein
PET images from the part-specific image reconstruction unit and whole-body-specific image reconstruction unit are combined to output a composite image.

24. A proximity imaging type PET apparatus system, comprising:
a part-specific PET detector disposed in proximity to a specific part of a measurement target;
a part-specific radiation position computing unit for performing position computing based on an output from the part-specific PET detector and then outputting single event data;
a part-specific coincidence circuit for finding out two pieces of single event data which are a pair of annihilation radiations and outputting the resulting data as coincidence data;
a part-specific data collecting unit;
a whole-body PET detector capable of radiographing a whole body of the measurement target;
a whole-body-specific radiation position computing unit for performing position computing based on an output from the whole-body PET detector and outputting single event data;
a whole-body-specific coincidence circuit for finding out two pieces of single event data which are a pair of annihilation radiations and outputting the resulting data as coincidence data;
a whole-body-specific data collecting unit; and
an image reconstruction unit for reconstructing an image based on outputs from the part-specific data collecting unit and the whole-body-specific data collecting unit.

25. A proximity imaging type PET apparatus system, comprising:
a part-specific PET detector disposed in proximity to a specific part of a measurement target;
a part-specific radiation position computing unit for performing position computing based on an output from the part-specific PET detector and then outputting single event data;
a whole-body PET detector capable of radiographing a whole body of the measurement target;
a whole-body-specific radiation position computing unit for performing position computing based on an output from the whole-body PET detector and outputting single event data;
a coincidence unit for finding out two pieces of single event data, which are a pair of annihilation radiations, from data into which pieces of single event data provided by the part-specific radiation position computing unit and the whole-body-specific radiation position computing unit are combined, and outputting the resulting data as coincidence data;
a data collecting unit; and
an image reconstruction unit for reconstructing an image based on an output from the data collecting unit.

26. A proximity imaging type PET apparatus system, comprising:
a part-specific PET detector disposed in proximity to a specific part of a measurement target;
a part-specific radiation position computing unit for performing position computing based on an output from the part-specific PET detector and then outputting single event data;
a part-specific data collecting unit for saving the single event data;
a whole-body PET detector capable of radiographing a whole body of the measurement target;
a whole-body-specific radiation position computing unit for performing position computing based on an output from the whole-body PET detector and outputting single event data;
a whole-body-specific data collecting unit for saving the single event data;
a coincidence unit for finding out two pieces of single event data, which are a pair of annihilation radiations, from data into which pieces of single event data provided by the part-specific data collecting unit and the whole-body-specific data collecting unit are combined, and outputting the resulting data as coincidence data; and
an image reconstruction unit for reconstructing an image based on an output from the coincidence unit.

27. A proximity imaging type PET apparatus system, comprising:
a part-specific PET detector disposed in proximity to a specific part of a measurement target;
a part-specific radiation position computing unit for performing position computing based on an output from the part-specific PET detector and then outputting single event data;
a whole-body PET detector capable of radiographing a whole body of the measurement target;
a whole-body-specific radiation position computing unit for performing position computing based on an output from the whole-body PET detector and outputting single event data;
a data collecting unit for combining and saving the two types of single event data;
a coincidence unit for finding out, from combined data, two pieces of single event data which are a pair of annihilation radiations and outputting the resulting data as coincidence data; and
an image reconstruction unit for reconstructing an image based on an output from the coincidence unit.
